(19) United States
(12) Patent Application Publication
(10) Pub. No.: US 2023/0404780 A1
(43) Pub. Date: Dec. 21, 2023

(54) STENT HAVING ASSISTANT EXTENSION PORTION

(71) Applicant: SG Biomedical Co., Ltd., Hsinchu County (TW)

(72) Inventors: PING-TUN TENG, Hsinchu County (TW); MAO TING, Hsinchu County (TW); HUNG-WEN FANG, Hsinchu County (TW); CHIUNG-JU LIN, Hsinchu County (TW); CHIEN-HSING WU, Hsinchu County (TW)

(21) Appl. No.: 18/210,315

(22) Filed: Jun. 15, 2023

(30) Foreign Application Priority Data
Jun. 20, 2022 (TW) 111122864

(51) Int. Cl. A61F 2/89

(52) U.S. Cl. CPC A61F 2/89; A61F 2250/0007; A61F 2250/0096

(57) ABSTRACT

A stent having assistant extension portion includes a plurality of ring layers, connecting layer, and assistant extension portion. Each ring layer has a plurality of first crowns connected to each other. The connecting layer has the first crown and a second crown connected to each other. The connecting layer and the ring layers are connected to each other to form a stent body, and the connecting layer is located at one end of the stent body. The assistant extension portion has a fixed end and a free end, the fixed end of the assistant extension portion is connected to the second crown, and the free end of the assistant extension portion has at least one hole.

### CROSS REFERENCE TO RELATED APPLICATION

All related applications are incorporated by reference. The present application is based on, and claims priority from, Taiwan Application Serial Number 111122864, filed on Jun. 20, 2022, the disclosure of which is hereby incorporated by reference herein in its entirety.

### TECHNICAL FIELD

The present invention relates to a stent, in particular to a stent having assistant extension portion.

### BACKGROUND

If the patient needs hemodialysis, the doctors need to perform arteriovenous surgery for the patient first. When performing vascular surgery, doctors need to place stents into the patient's veins first, and then connect the veins and arteries with sutures. However, it is difficult to connect veins and arteries through suturing, which prolongs the operation time of arteriovenous surgery.

In addition, when a vein is connected to an artery, blood from the artery flows into the vein to the arterialization of venous blood. However, when arterial blood flows to veins, the problem of the angle between each other will produce the so-called turbulent flow and pressure difference and will make the blood flow between veins and arteries unsmooth, reducing the success rate of arteriovenous surgery.

The stents implanted in veins may be deflected due to blood flow or external forces generated by patient activities, away from the arteriovenous junction. Generally, regular observation after arteriovenous surgery depends on the markers on the stent to confirm the movement to ensure the vein's position and whether the implantation is successful. If the stent is displaced due to external force, it is not conducive to regular follow-up observation after surgery.

### SUMMARY

According to an embodiment of the present invention, the present invention proposes a stent having assistant extension portion, which comprises a plurality of ring layers, a connecting layer, and an assistant extension portion. Each ring layer has a plurality of first crowns connected to each other. The connecting layer has the plurality of first crowns and a second crown connected to each other. The connecting layer and the plurality of ring layers are connected to each other to form a stent body. The connecting layer is located at one end of the stent body. The assistant extension portion has a fixed end and a free end, the fixed end of the assistant extension portion is connected to the second crown, and the free end of the assistant extension portion has at least one hole.

In one embodiment, a diameter of the hole is 0.4 mm˜1 mm.

In one embodiment, a width of the second crown is greater than or equal to the width of the first crown.

In one embodiment, two corners of the free end of the assistant extension portion are rounded.

In one embodiment, a section of the stent body forms a reference circle, a section of the assistant extension portion forms an arc, and a ratio of the arc length of the arc to a circumference of the reference circle is 1/4˜1.

In one embodiment, a section of the stent body forms a reference circle, and a diameter of the reference circle is 2 mm˜20 mm.

In one embodiment, a section of the assistant extension portion forms an arc, and an arc length of the arc is 0.5 mm˜31.4 mm.

In one embodiment, the assistant extension portion comprises an extension body and a connection portion connected to each other, the extension body is connected to the second crown through the connection portion, and a width of the connection portion is smaller than the width of the extension body to form two grooves in two sides of the assistant extension portion.

In one embodiment, a width of the groove gradually increases from the bottom of the groove to the top of the groove.

In one embodiment, a shortest distance from the central point of the hole to the edge of the extension body is 0.5 mm˜1 mm.

In one embodiment, a ratio of a width of the connection portion to a width of the extension portion body is 0.5˜1.

In one embodiment, one of the first crowns of each ring layer is connected to one of the first crowns of adjacent ring layers through a bridging portion.

Based on the above, according to the stent having assistant extension portion of the present invention, it may have one or more of the following advantages:
(1) In one embodiment of the present invention, the stent has an assistant extension portion, and the assistant extension portion has at least one hole, so the doctor can expose the free end of the assistant extension portion from the opening of the vein when performing an arteriovenous surgery, and use the hole as a reference point to pass the surgical needle through the hole for suturing. Therefore, the time for doctors to perform arteriovenous surgery can be greatly shortened.
(2) In one embodiment of the present invention, the assistant extension portion of the stent comprises an extension body and a connection portion connected to each other. The extension body is connected to the second crown through the connection portion, and the width of the connection portion is smaller than the width of the extension body. The above-mentioned structural design allows the connecting portion to be used as a bending point so that the doctor can use the connecting portion as a bending point to bend the stent. When placing the stent into the vein for arteriovenous surgery, the vein and the artery have certain angles degree between them, so that the doctor can perform the arteriovenous catheter operation more smoothly, so the time for the doctor to perform the arteriovenous surgery can be further shortened.
(3) In one embodiment of the present invention, the stent body of the stent has a connecting layer, and the connecting layer has a plurality of the first crowns connected to each other and a second crown wider than the first crown. The stent body is connected to the connecting portion through the second crown and the assistant extension portion. In addition, both the second crown and the assistant extension portion (the extension body) have a certain radian. The combination of the second crown of the connecting layer and the connection portion of the assistant extension portion can provide high mechanical strength, so after the arteriovenous surgery is completed, the connection between the vein and the artery will not be narrowed maturation due to the excessive moving distance and torsion during the surgery to improve the surgical effect of the arteriovenous surgery greatly.
(4) In one embodiment of the present invention, the two corners of the free end of the assistant extension portion of the stent are rounded, which can prevent the assistant extension portion from irritating the vein or artery to improve the effectiveness of the arteriovenous surgery.
(5) In one embodiment of the present invention, the stent has an assistant extension portion, and the assistant extension portion has at least one hole. The doctor can use the hole as a reference point to pass the surgical needle through the hole for suturing so that the assistant extension portion can be fixed on the arteriovenous junction to attach the inner wall of the blood vessel, so the deviation of the stent due to external force can be avoided and the stent can be implanted in an ideal position. At the same time, because the free end of the assistant extension portion is fixed at the arteriovenous junction, during postoperative observation and tracking, the positioning accuracy of the marker can be further improved under the irradiation of the imaging device, reducing the time to find the previous surgical location and shortening the time of the overall postoperative observation.
(6) In one embodiment of the present invention, the structure design of the stent is simple, so the desired effect can be achieved without greatly increasing the cost, which is of great commercial value.

### BRIEF DESCRIPTION OF THE DRAWINGS

FIG. 1 is a three-dimensional diagram of the stent having assistant extension portion of the present invention.

FIG. 2 is a top diagram of the stent having assistant extension portion of the present invention.

FIG. 3 is a partial enlarged diagram of the stent having assistant extension portion of the present invention.

FIG. 4 is a schematic diagram of the stent having assistant extension portion of the present invention.

FIG. 5 is the first schematic diagram of the usage state of the stent having assistant extension portion of the present invention.

FIG. 6 is the second schematic diagram of the usage state of the stent having assistant extension portion of the present invention.

### DETAILED DESCRIPTION

The following will refer to related drawings to illustrate embodiments of the stent having assistant extension portion according to the present invention. For the sake of clarity and convenience of illustration, the size and proportion of each component in the drawings may be exaggerated or reduced. In the following description and/or claims, when it is mentioned that an element is “connected” or “coupled” to another element, it may be directly connected or coupled to the other element or there may be an intervening element. When “directly connected” or “directly coupled” to another element, there are no intervening elements present, and other words used to describe the relationship between elements or layers should be interpreted in the same manner. To facilitate understanding, the same components in the following embodiments are described with the same symbols.

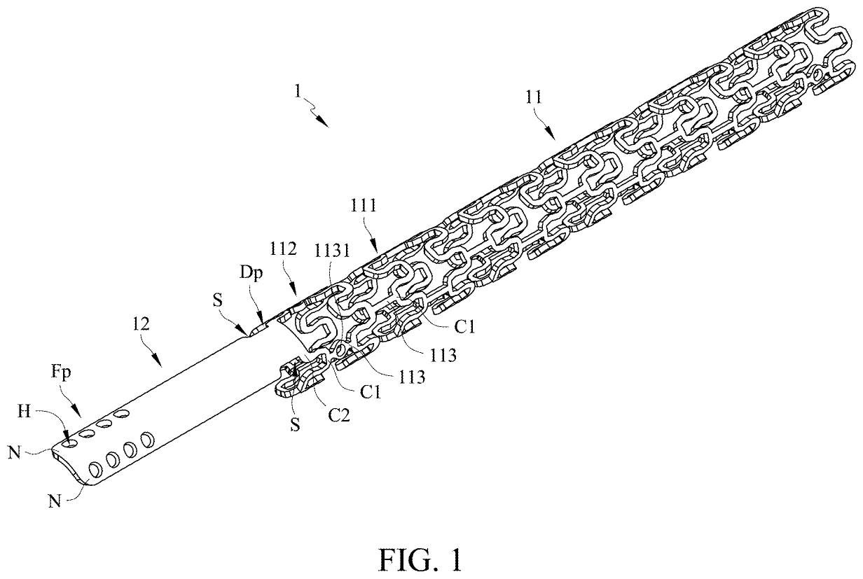

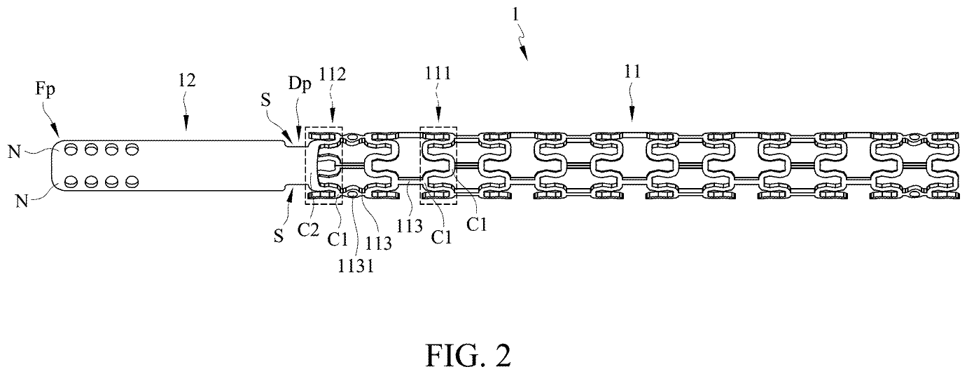

Please refer to FIG. 1 and FIG. 2. FIG. 1 and FIG. 2 are the three-dimensional diagram and the top view diagram of the stent having assistant extension portion of the present invention. As shown in the figures, the stent 1 includes a plurality of ring layers 111, a connecting layer 112, and an assistant extension portion 12.

The above-mentioned plurality of ring layers 111 and connecting layer 112 are connected to each other to form a stent body 11. The connecting layer 112 is located at one end of the stent body 11.

Each ring layer 111 has a plurality of first crown C1 connected to each other. In this embodiment, each ring layer 111 has eleven first crown C1. The three first crowns C1 of each ring layer 111 are respectively connected to the corresponding three first crowns C1 of the adjacent ring layers 111 through three bridging portions 113. In addition, at least one bridging portion 113 between the ring layer 111 at the other end of the stent body 11 and the adjacent ring layer 111 may also has a marker hole 1131 and a marker is disposed in the marker hole 1131. Certainly, this embodiment is only an example, and the quantity and structure of the ring layer 111, the first crown C1, and the bridging portion 113 can be changed according to actual needs.

The connecting layer 112 has the plurality of the first crowns C1 and the second crown C2 connected to each other. The three first crowns C1 of each connecting layer 112 are respectively connected to the corresponding three first crowns C1 of the adjacent ring layers 111 through three bridging portions 113. In the same way, at least one bridging portion 113 between the connecting layer 112 and the adjacent ring layer 111 may also has a marker hole 1131 and a marker is disposed in the marker hole 1131. In this embodiment, the width of the second crown C2 is greater than the width of the first crown C1. In another embodiment, the width of the second crown C2 may also be equal to the width of the first crown C1. For example, the ratio of the width of the second crown C2 to the width of the first crown C1 maybe 2˜1.

The assistant extension portion 12 has a fixed-end Dp and a free-end Fp. The fixed end Dp of the assistant extension portion 12 is connected to the second crown C2, and the free end Fp of the assistant extension portion 12 has a plurality of holes H. The two corners N of the free end Fp of the assistant extension portion 12 are rounded to prevent the assistant extension portion 12 from irritating the vein or artery during the arteriovenous surgery.

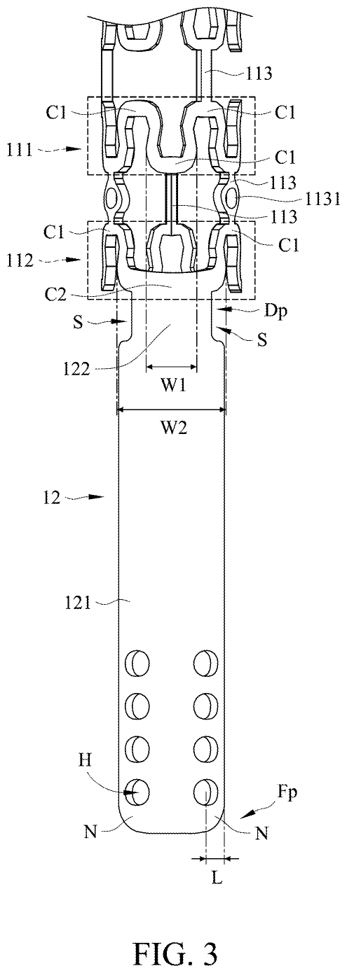

Please refer to FIG. 3. FIG. 3 is a partial enlarged diagram of the stent having assistant extension portion of the present invention, and please also refer to FIG. 1 and FIG. 2. As shown in the figure, the assistant extension portion 12 includes an extension body 121 and a connecting portion 122. The extension body 121 is connected to the connection portion 122, and the extension body 121 is connected to the second crown C2 through the connection portion 122. In this embodiment, the width of the connecting portion 122 is smaller than the width of the extension body 121 and also smaller than the width of the second crown C2. Therefore, two grooves S are formed on two sides of the assistant extension portion 12. The width of groove S gradually increases from the bottom of the groove S to the top of the groove S. In one embodiment, the ratio of the width of the connection portion 122 to the width of the extension body 121 may be which is slightly smaller than the width of the second crown C2. In another embodiment, the ratio of the width of the connecting portion 122 to the width of the extension body 121 maybe 0.7. In an embodiment, the width of the connection portion 122 may also be equal to the width of the extension body 121 and the width of the second crown C2. These embodiments are only examples, and the structures of the above components can be adjusted according to actual needs.

As mentioned above, the width of the connecting portion 122 is smaller than the width of the extension body 121 and also smaller than the width of the second crown C2 to form two grooves S in the two sides of the assistant extension portion 12. Therefore, when the doctor performs an arteriovenous surgery, the stent 1 can be placed into the vein and the connecting portion 112 is used as a bending point so that the stent 1 can be bent at a specific angle. In this way, there can be an appropriate angle between the vein and the artery, so that the doctor can smoothly perform the arteriovenous surgery.

The holes H are disposed on the extension body 121. In this embodiment, the number of these holes H maybe eight. Wherein, the shortest distance L from the center point of any hole H to the edge of the extension body 121 is 0.5 mm˜1 mm. In addition, the diameter of the hole H maybe 0.4 mm˜1 mm. In one embodiment, the diameter of the hole H maybe 0.45 mm. This embodiment is only an example, and the number and structure of hole H can be adjusted according to actual needs.

It can be known from the above that the stent 1 has an assistant extension portion 12, and the extension body 121 of the assistant extension portion 12 has a plurality of holes H. Therefore, the doctor can expose the free end Fp of the assistant extension portion 12 from the opening of the vein when performing an arteriovenous surgery, and use the above-mentioned the plurality of holes H as reference points to pass the surgical needle through the hole H for suturing. Therefore, when doctors perform arteriovenous surgery, the above-mentioned plurality of holes H can be used as clear reference points, so that doctors can suture veins and arteries quickly and efficiently to short the operation time greatly.

In addition, the connecting layer 112 has a plurality of first crowns C1 and second crown C2. The width W2 of the second crown C2 is greater than the width W1 of the first crown C1, and the extension body 121 of the assistant extension portion 12 is connected to the second crown C2 through the connecting portion 122. Both the second crown C2 and the assistant extension portion 12 (the extension body 121) have certain radian. Therefore, the combination of the second crown C2 of the connecting layer 112 and the connection portion of the assistant extension portion 12 can provide higher mechanical strength. Therefore, after the arteriovenous surgery is completed, the connection between the vein and the artery will not be narrowed maturation due to the excessive movement distance and torsion during the operation, so the surgical effect of the arteriovenous surgery can be effectively improved.

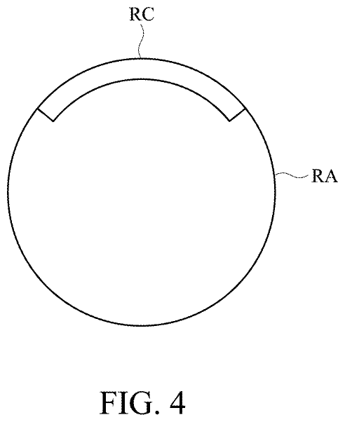

Please refer to FIG. 4. FIG. 4 is a schematic diagram of the stent having assistant extension portion of the present invention. As shown in the figure, the section of the stent body 11 can be used as a reference circle RC, and the diameter of the reference circle can be 2 mm˜20 mm. The section of the assistant extension portion 12 can form an arc RA, and the arc length of the arc can be 0.5 mm to 31.4 mm. In one embodiment, the ratio of the arc length of the arc RA to the circumference of the reference circle is 1/4˜1. In this embodiment, the ratio of the arc length of the arc RA to the circumference of the reference circle is 1/3.

Certainly, this embodiment is only for illustration and not limiting the scope of the present invention, and equivalent modifications or changes made according to the stent having assistant extension portion 1 of this embodiment should still be included in the patent scope of the present invention.

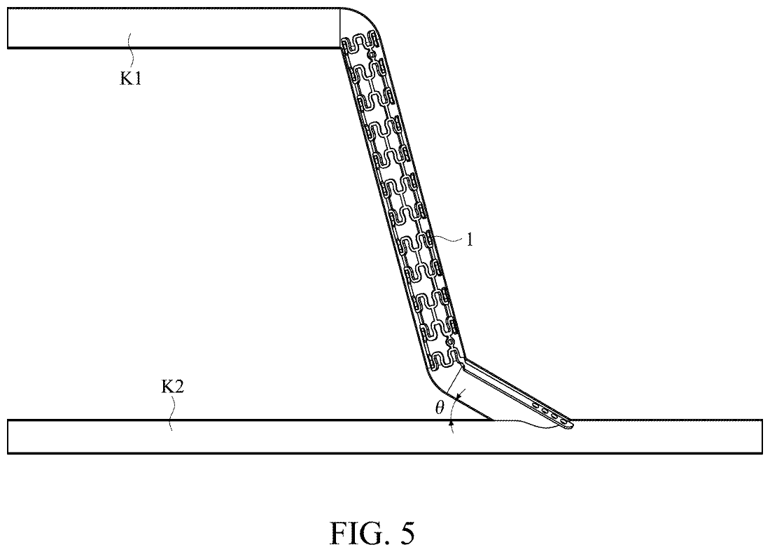

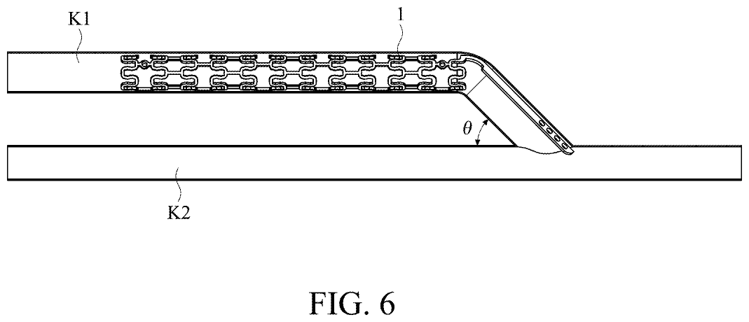

Please refer to FIG. 5 and FIG. 6. FIG. 5 and FIG. 6 are the first schematic diagram and the second schematic diagram of the use state of the stent having assistant extension portion of the present invention, and please refer to FIG. 1, FIG. 2 and FIG. 3 at the same time. As shown in FIG. 5, when the doctor performs an arteriovenous surgery to suture the vein K1 and the artery K2, the stent 1 can be inserted into the vein K1 and the connecting portion 112 is used as a bending point to make the stent 1 to be bent at a specific angle degree A. In addition, the doctor can use the plurality of holes H as reference points to pass the surgical needle through the hole H for suturing. In this way, doctors can perform the arteriovenous surgery quickly and efficiently.

As shown in FIG. 6, the combination of the second crown C2 of the connecting layer 112 of the stent body 11 and the connection portion 122 of the assistant extension portion 12 can provide higher mechanical strength. Therefore, after the arteriovenous surgery is completed, the connection between the vein and the artery will not be narrowed maturation due to the excessive movement distance and torsion during the surgery, so the surgical effect of the arteriovenous surgery can be effectively improved.

Certainly, this embodiment is only for illustration and not limiting the scope of the present invention, and equivalent modifications or changes made according to the stent having assistant extension portion 1 of this embodiment should still be included in the patent scope of the present invention.

To sum up, according to the embodiment of the present invention, the stent has an assistant extension portion, and the assistant extension portion has at least one hole, so the doctor can expose the free end of the assistant extension portion from the opening of the vein when performing an arteriovenous surgery, and use the hole as a reference point to pass the surgical needle through the hole for suturing. Therefore, the time for doctors to perform the arteriovenous surgery can be greatly shortened.

According to an embodiment of the present invention, the assistant extension portion of the stent includes an extension body and a connection portion connected to each other. The extension body is connected to the second crown through the connection portion, and the width of the connection portion is smaller than the width of the extension body. The above-mentioned structural design allows the connecting part to be used as a bending point so that the doctor can use the connecting part as a bending point to bend the stent when placing the stent into the vein for arteriovenous surgery to form a certain angle degree between the vein and the artery. The doctor can perform the arteriovenous surgery more smoothly, so the time for the doctor to perform the arteriovenous surgery can be further shortened.

Furthermore, according to an embodiment of the present invention, the stent body of the stent has a connecting layer, and the connecting layer has a plurality of first crowns connected to each other and a second crown with a width larger than the first crown. The stent body is connected to the assistant extension portion through the second crown. In addition, the second crown and the assistant extension portion (the extension body) both have a certain radian. The combination of the second crown of the connecting layer and the connection portion of the assistant extension portion can provide high mechanical strength, so after the arteriovenous surgery is completed, the connection between the vein and the artery will not be narrowed maturation due to the excessive moving distance and torsion during the surgery to improve the surgical effect of arteriovenous surgery greatly.

In addition, according to the embodiment of the present invention, the two corners of the free end of the assistant extension portion of the stent are rounded to prevent the assistant extension portion from irritating the vein or artery, so the surgical effect of the arteriovenous surgery can be further improved.

In addition, according to an embodiment of the present invention, the stent has an assistant extension portion, and the assistant extension portion has at least one hole. The doctor can use the hole as a reference point to pass a surgical needle through the hole for suturing, so that the assistant extension portion can be fixed on the arteriovenous junction to attach the inner wall of the blood vessel, so the deviation of the stent due to external force can be avoided and the stent can be implanted in an ideal position. At the same time, because the free end of the assistant extension portion is fixed at the arteriovenous junction, during postoperative observation and tracking, the positioning accuracy of the marker can be further improved under the irradiation of the imaging device, reducing the time to find the previous surgical location and shortening the time of the overall postoperative observation.

Furthermore, according to the embodiments of the present invention, the structure design of the stent is simple, so the desired effect can be achieved without greatly increasing the cost, which is of great commercial value.

It can be seen that the present invention has indeed achieved the desired effect after breaking through the prior art, and it is not easy for those who are familiar with this technology to think about it. The non-obviousness and the industrially applicability of the present invention obviously meet the requirements for patent application. I filed a patent application by law, and I sincerely ask the patent bureau to approve this invention patent application to encourage the creation, and I am grateful for it.

The above descriptions are illustrative only, not restrictive. Any other equivalent modifications or changes made without departing from the spirit and scope of the present invention shall be included in the scope of the appended patent application.

What is claimed is:

1. A stent having assistant extension portion, comprising:
a plurality of ring layers, each of the ring layers having a plurality of first crowns connected to each other; and
a connecting layer having the plurality of first crowns and a second crown connected to each other, wherein the connecting layer and the plurality of ring layers are connected to each other to form a stent body, the connecting layer is located at one end of the stent body;
an assistant extension portion having a fixed end and a free end, the fixed end of the assistant extension portion being connected to the second crown, and the free end of the assistant extension portion having at least one hole.

2. The stent having assistant extension portion of claim 1, wherein a diameter of the hole is 0.4 mm˜1 mm.

3. The stent having assistant extension portion of claim 1, wherein a width of the second crown is greater than or equal to a width of the first crown.

4. The stent having assistant extension portion of claim 1, wherein two corners of the free end of the assistant extension portion are rounded.

5. The stent having assistant extension portion of claim 1, wherein a section of the stent body forms a reference circle, a section of the assistant extension portion forms an arc and a ratio of an arc length of the arc to a circumference of the reference circle is 1/4˜1.

6. The stent having assistant extension portion of claim 1, wherein a section of the stent body forms a reference circle, and a diameter of the reference circle is 2 mm˜20 mm.

7. The stent having assistant extension portion of claim 1, wherein a section of the assistant extension portion forms an arc, and an arc length of the arc is 0.5 mm˜31.4 mm.

8. The stent having assistant extension portion of claim 1, wherein the assistant extension portion comprises an extension body and a connecting portion connected to each other, the extension body is connected to the second crown through the connecting portion, and a width of the connecting portion is smaller than a width of the extension body to form two grooves in two sides of the assistant extension portion.

9. The stent having assistant extension portion of claim 8, wherein a width of the groove gradually increases from a bottom of the groove to a top of the groove.

10. The stent having assistant extension portion of claim 8, wherein a shortest distance from a central point of the hole to an edge of the extension body is 0.5 mm˜1 mm.

11. The stent having assistant extension portion of claim 1, wherein a ratio of a width of the connection portion to a width of the extension portion body is 0.5˜1.

12. The stent having assistant extension portion of claim 1, wherein one of the first crowns of each of the ring layers is connected to one of the first crowns of the ring layer adjacent thereto through a bridging portion.

13. The stent having assistant extension portion of claim 12, wherein the bridging portion has a marker hole and a marker is disposed in the marker hole.

14. The stent having assistant extension portion of claim 1, wherein one of the first crowns of the connecting layer is connected to one of the first crowns of the ring layers adjacent thereto through a bridging portion.

15. The stent having assistant extension portion of claim 14, wherein the bridging portion has a marker hole and a marker is disposed in the marker hole.
